(19) United States
(12) Patent Application Publication
(10) Pub. No.: US 2004/0029865 A1
(43) Pub. Date: Feb. 12, 2004

(54) Bis-arylsulfones

(76) Inventors: Brad A. Acker, Kalamazoo, MI (US); Robert Shih-Liang Chao, Santa Clara, CA (US); Michael Hawley, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/437,478

(22) Filed: May 14, 2003

Related U.S. Application Data
(60) Provisional application No. 60/382,685, filed on May 23, 2002.

(51) Int. Cl. A61K 31/55; C07D 243/08

(52) U.S. Cl. 514/218; 540/575

(57) ABSTRACT

The present invention provides pharmaceutically active compounds useful for the treatment of diseases or disorders of the central nervous system.

### CROSS REFERENCE TO RELATED APPLICATIONS

This application claims the benefit of U.S. provisional application Serial No. 60/382,685 filed on May 23, 2002, under 35 USC 119(e)(i), which is incorporated herein by reference in its entirety.

### FIELD OF THE INVENTION

The present invention relates to bis-arylsulfone derivatives, and more specifically, relates to bis-arylsulfone compounds of formula I described herein below. These compounds are 5-HT receptor ligands and are useful for treating diseases wherein modulation of 5-HT activity is desired.

### BACKGROUND OF THE INVENTION

Serotonin has been implicated in a number of diseases and conditions that originate in the central nervous system. These include diseases and conditions related to sleeping, eating, perceiving pain, controlling body temperature, controlling blood pressure, depression, anxiety, schizophrenia, and other bodily states. Serotonin also plays an important role in peripheral systems, such as the gastrointestinal system, where it has been found to mediate a variety of contractile, secretory, and electrophysiologic effects.

As a result of the broad distribution of serotonin within the body, there is a tremendous interest in drugs that affect serotonergic systems. In particular, agonists, partial agonists and antagonists are of interest for the treatment of a wide range of disorders, including anxiety, depression, hypertension, migraine, obesity, compulsive disorders, schizophrenia, autism, neurodegenerative disorders (e.g. Alzheimer's disease, Parkinsonism, and Huntington's chorea), and chemotherapy-induced vomiting.

### SUMMARY OF THE INVENTION

The present invention provides compounds of formula I:

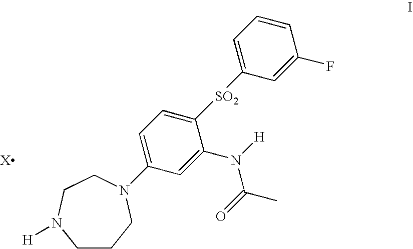

wherein X is selected from HCI, HOC(O)CF₃, cis-HO(CO)—CH═CH—(CO)OH, and

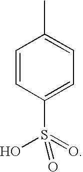

In another aspect, the invention further provides a pharmaceutical composition including a compound of formula I and a pharmaceutically acceptable carrier. In certain embodiments of this aspect of the invention, the composition includes a therapeutically effective amount of a compound of formula I.

In another aspect, the present invention further provides a method for treating or preventing diseases or disorders of the central nervous system comprising administering a therapeutically effective amount of a compound of formula I to a mammal. In certain embodiments, the compound of formula I may be used to prepare a medicament for treating or preventing diseases or disorders of the central nervous system.

In yet another aspect, the invention provides a method for treating a disease or condition in a mammal wherein a 5-HT receptor is implicated and modulation of a 5-HT function is desired comprising administering to the mammal a therapeutically effective amount of a compound of formula I.

Embodiments of each aspect of the invention may include one or more of the following features. The 5-HT receptor is a 5-HT₆ receptor. The diseases or disorders of the central nervous system include anxiety, stress related disorders, depression, obesity. The value of X is HCl. The value of X is cis-HO(CO)—CH═CH—(CO)OH. The value of X is HOC(O)CF₃. The value of X is cis-HO(CO)—CH═CH—(CO)OH and the compound is crystalline. The compound is crystalline form I of N-{5-(1,4-diazepan-1-yl)-2-[(3-fluorophenyl)sulfonyl] phenyl} acetamide maleate. The compound is crystalline form II of N-{5-(1,4-diazepan-1-yl)-2-[(3-fluorophenyl)sulfonyl]phenyl}acetamide maleate. The value of X is

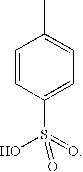

Advantageously, the compounds of formula I are stable salts of N-[5-(1,4-diazepan-1-yl)-2-[(3-fluorophenyl)sulfonyl]phenyl]acetamide. Unexpectedly, the compounds of formula I exhibit lower moisture absorption, stability at high relative humidity, and stability at high temperatures, relative to other pharmaceutical salts of N-[5-(1,4-diazepan-1-yl)-2-[(3-fluorophenyl)sulfonyl]phenyl]acetamide. Compounds possessing lower moisture absorption, stability at high relative humidity, and stability at high temperatures are easier to formulate, such as with wet granulation techniques. As a result, pharmaceutical compositions including the compounds of formula I have stable, well characterized physical properties, e.g., solubility, melting point, etc.

### DETAILED DESCRIPTION OF THE DRAWINGS

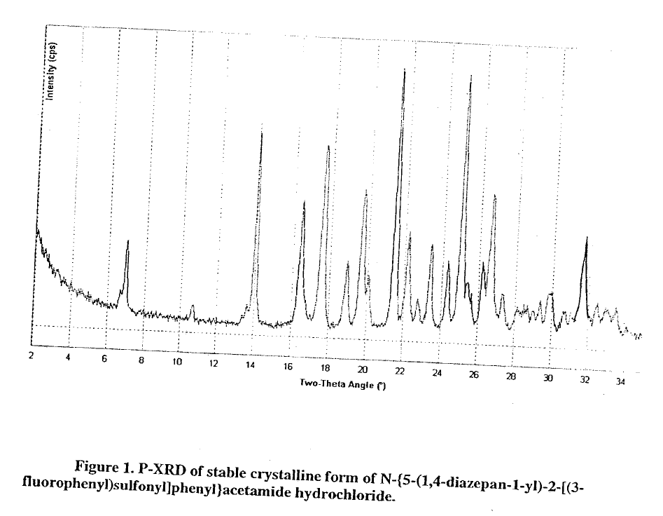

FIG. 1 shows a plot of a powder X-ray Diffraction spectra of N-{5-(1,4-diazepan-1-yl)-2-[(3-fluorophenyl)sulfonyl]phenyl}acetamide hydrochloride salt as the stable crystalline form.

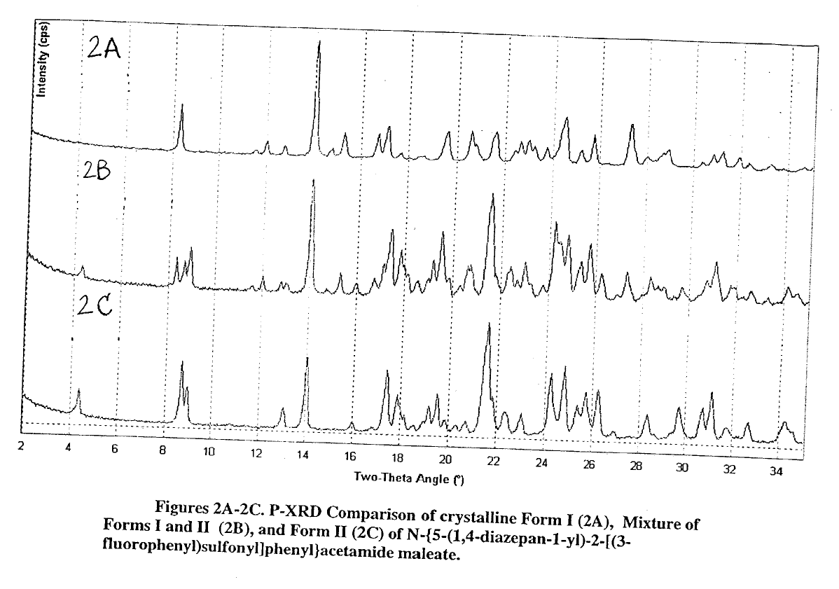

FIGS. 2A, 2B, and 2C show plots of powder X-ray Diffraction spectra of N-{5-(1,4-diazepan-1-yl)-2-[(3-fluorophenyl)sulfonyl]phenyl}acetamide maleate as crystalline form I (FIG. 2A), mixture of crystalline forms I and II (FIG. 2B), and crystalline form II (FIG. 2C).

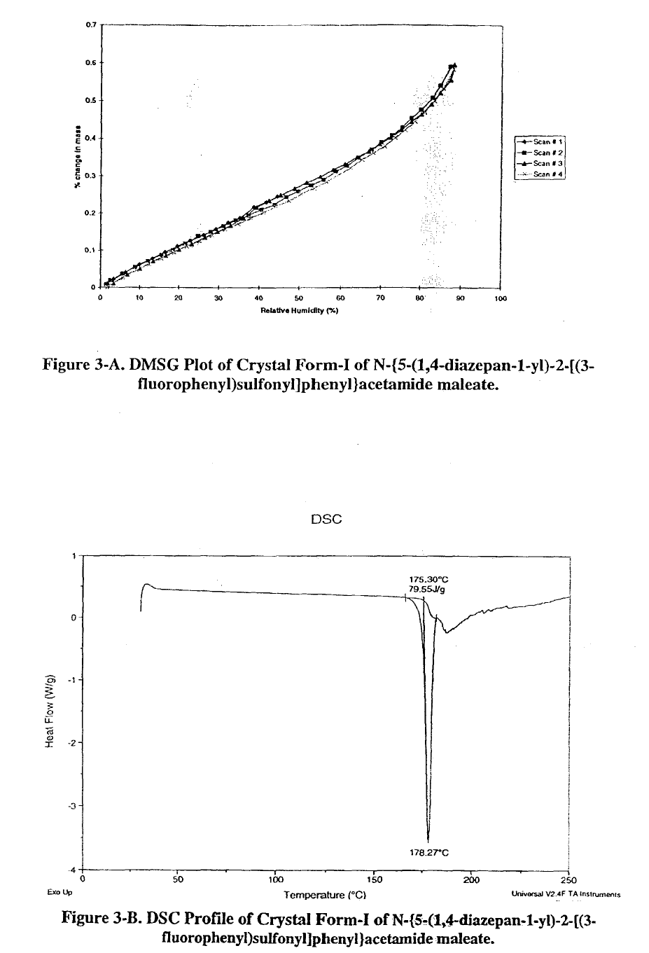

FIG. 3 shows plots of Dynamic moisture sorption gravimetry and Differential scanning calorimetry for crystalline form I of N-{5-(1,4-diazepan-1-yl)-2-[(3-fluorophenyl)sulfonyl]phenyl}acetamide maleate.

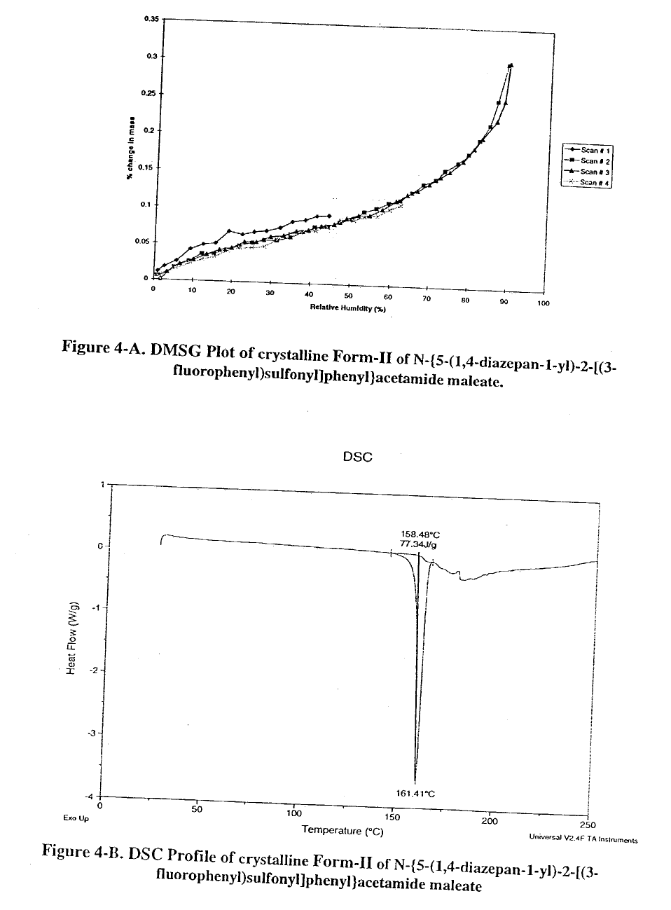

FIG. 4 shows plots of Dynamic moisture sorption gravimetry and Differential scanning calorimetry for crystalline form II of N-{5-(1,4-diazepan-1-yl)-2-[(3-fluorophenyl)sulfonyl]phenyl}acetamide maleate.

### DETAILED DESCRIPTION OF THE INVENTION

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours, “rt” for room temperature, and etc.).

Mammal denotes human and animals.

The physical characteristics of each example were tested by recording X-ray Diffraction (XRD) Analysis, Differential scanning calorimetry (DSC), and Dynamic moisture sorption gravimetry (DMSG).

It is to be understood that the present invention encompasses any racemic mixture, enantiomerically pure, optically-active, polymorphic, tautomeric, or stereoisomeric form, or mixture thereof, of a compound of the invention, which possesses the useful properties described herein.

The major classes of serotonin receptors (5-HT₁₋₇) contain fourteen to eighteen separate receptors that have been formally classified. See Glennon, et al., Neuroscience and Behavioral Reviews, 1990, 14, 35; and D. Hoyer, et al. Pharmacol. Rev. 1994, 46, 157-203.

There is currently a need for pharmaceutical agents that are useful to treat diseases and conditions that are associated with 5-HT receptors. In particular, there is a need for agents that can selectively bind to individual receptor subtypes (e.g. receptor-specific agonists or antagonists); such agents would be useful as pharmaceutical agents, or would be useful to facilitate the study of the 5-HT receptor family, or to aid in the identification of other compounds that selectively bind to the specific 5-HT receptors.

For example, the 5-HT₆ receptor is identified in 1993 (Monsma et al. Mol. Pharmacol. 1993, 43, 320-327 and Ruat, M. et al. Biochem. Biophys. Res. Com. 1993, 193, 269-276). Several antidepressants and a typical antipsychotics bind to the 5-HT₆ receptor with high affinity and this binding may be a factor in their profile of activities (Roth et al. J. Pharm. Exp. Therapeut. 1994, 268, 1403-1410; Sleight et al. Exp. Opin. Ther. Patents 1998, 8, 1217-1224; Bourson et al. Brit. J. Pharm. 1998, 125, 1562-1566; Boess et al. Mol. Pharmacol. 1998, 54, 577-583; Sleight et al. Brit. J. Pharmacol. 1998, 124, 556-562). In addition, the 5-HT₆ receptor has been linked to generalized stress and anxiety states (Yoshioka et al. Life Sciences 1998, 17/18, 1473-1477). Together these studies and observations suggest that compounds that antagonize the 5-HT₆ receptor will be useful in treating disorders of the central nervous system.

Generally, compounds of the present invention are 5-HT ligands. In particular, they can selectively bind to the 5-HT₆ receptor (e.g. receptor-specific agonists or antagonists). Thus, they are useful for treating diseases wherein modulation of 5-HT activity, specifically 5-HT₆ activity, is desired. Therefore, the compounds of this invention are useful for the treatment of diseases or disorders of the central nervous system. More specifically, for the treatment of any one or more of the following: psychosis, paraphrenia, psychotic depression, mania, schizophrenia, schizophreniform disorders, anxiety, migraine headache, drug addiction, convulsive disorders, personality disorders, post-traumatic stress syndrome, alcoholism, panic attacks, obsessive-compulsive disorders, and sleep disorders. The compounds of this invention are also useful to treat any one or more of the following: psychotic, affective, vegetative, and psychomotor symptoms of schizophrenia and the extrapyramidal motor side effects of other antipsychotic drugs. This last action will allow higher doses of antipsychotics to be used and thus greater antipsychotic efficacy to be obtained as a result of a reduction in side effects. The compounds of this invention are also useful in the modulation of eating behavior and thus are useful in treating excess weight and associated morbidity and mortality and also with eating disorders including anorexia and bulemia.

Diseases or disorders for which a compound of formula I may have activity include, but are not limited to the following: obesity, depression, schizophrenia, schizophreniform disorder, schizoaffective disorder, delusional disorder, a stress related disease (e.g. general anxiety disorder), panic disorder, a phobia, obsessive compulsive disorder, post-traumatic-stress syndrome, immune system depression, major depression, a stress induced problem with the urinary, gastrointestinal or cardiovascular system (e.g., stress incontinence), neurodegenerative disorders, autism, chemotherapy-induced vomiting, hypertension, migraine headaches, cluster headaches, sexual dysfunction in a mammal (e.g., a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, behavioral disturbance (including agitation in conditions associated with diminished cognition (e.g., dementia, mental retardation or delirium)), bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, movement disorder (e.g., Huntington's disease or Tardive Dyskinesia), oppositional defiant disorder, peripheral neuropathy, post-traumatic stress disorder, premenstrual dysphoric disorder, a psychotic disorder (brief and long duration disorders, psychotic disorder due to medical condition, psychotic disorder NOS), mood disorder (major depressive or bipolar disorder with psychotic features) seasonal affective disorder, a sleep disorder, cognitive disorders, iritable bowel syndrome, a specific developmental disorder, agitation disorder, selective serotonin reuptake inhibition (SSRI) “poop out” syndrome or a Tic disorder (e.g., Tourette's syndrome).

Compounds of the present invention can conveniently be administered in a pharmaceutical composition containing the compound in combination with a suitable excipient, the composition being useful in combating CNS diseases. Pharmaceutical compositions containing a compound appropriate for CNS diseases' use are prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically (including but not limited to surface treatment, transdermal application, and nasal application), intravaginally, orally, or rectally, depending on whether the preparation is used to treat a specific disease.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices such as the osmotic release type devices developed by the Alza Corporation under the OROS trademark.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The compound is conveniently administered in unit dosage form; for example, containing 1 to 1000 mg, conveniently 5 to 750 mg, most conveniently, 5 to 400 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compositions can be administered orally or parenterally at dose levels, calculated as the free base, of about 0.01 to 300 mg/kg mammal body weight, preferably 0.1 to 50 mg/kg of mammal body weight, more preferably 1.0 to 30 mg/kg of mammal body weight, and can be used in man in a unit dosage form, administered one to four times daily in the amount of 1 to 1000 mg per unit dose.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The exact regimen for administration of the compounds and compositions disclosed herein will necessarily be dependent upon the needs of the individual subject being treated, the type of treatment and, of course, the judgment of the attending practitioner. The compounds of the present invention can be administered to an animal in need of treatment. In most instances, this will be a human being, but the treatment of livestock and companion animals is also specifically contemplated as falling within the scope of the instant invention.

Generally, compounds of the invention are 5-HT ligands. The ability of a compound of the invention to bind or act at a 5-HT receptor, or to bind or act selectively at a specific 5-HT receptor subtype can be determined using in vitro and in vivo assays that are known in the art. As used herein, the term “bind selectively” means a compound binds at least 2 times, preferably at least 10 times, and more preferably at least 50 times more readily to a given 5-HT subtype than to one or more other subtypes. Preferred compounds of the invention bind selectively to one or more 5-HT receptor subtypes.

The ability of a compound of the invention to act as a 5-HT receptor agonist or antagonist can also be determined using in vitro and in vivo assays that are known in the art. All of the Example compounds provided above are 5-HT ligands, with the ability to displace>50% of a radiolabeled test ligand from one or more 5-HT receptor subtypes at a concentration of 1 μM. The procedures used for testing such displacement are well known.

General Procedure for the Preparation of Salts of N-[5-(1,4-diazepan-1-yl)-2-[(3-fluorophenyl)sulfonyl]phenyl]acetamide.

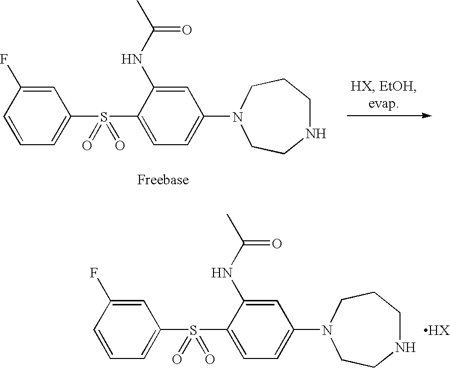

The salts are prepared by combining the acid and the freebase together in ethanol and allowing the solvent to evaporate slowly.

The freebase of N-[5-(1,4-diazepan-1-yl)-2-[(3-fluorophenyl)sulfonyl]phenyl]acetamide is obtained as follows (Freebase): A mixture of N-{5-(1,4-diazepan-1-yl)-2-[(3-fluorophenyl)sulfonyl]phenyl}acetamide methanesulfonate in CH₂Cl₂ was washed with 1 N NaOH until basic (pH paper). The organic layer was then dried (MgSO₄), filtered and concentrated to a sticky foam. The foam was diluted with ethanol to produce a known concentration of freebase in ethanol (0.1-0.33 M). This solution was transferred to a test-tube and the desired organic or inorganic acid was added. (Note: The acids were generally prepared as standard solutions using ethanol in volumetric flasks. In some cases, however, the acids were simply added to the vessel.) The mixtures were then diluted and warmed in an attempt to dissolve any solids that may have formed. Cooling and slow evaporation produced the desired crystalline solids, which were filtered and dried.

The invention also includes isotopically-labeled compounds, which are identical to those recited in Formula I but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹³N, ¹⁵O, and ¹⁸F. Compounds of the present invention and pharmaceutically acceptable salts and prodrugs of said compounds that contain the aforementioned isotopes and/or other isotopes of other atoms are within the scope of the invention. Isotopically-labeled compounds of the present invention are useful in drug and/or substrate tissue distribution and target occupancy assays. For example, isotopically labeled compounds are particularly useful in SPECT (single photon emission computed tomography) and in PET (positron emission tomography).

Single-photon emission computed tomography (SPECT), acquires information on the concentration of isotopically labeled compounds introduced to a mammal's body. SPECT dates from the early 1960's, when the idea of emission traverse section tomography was introduced by D. E. Kuhl and R. Q. Edwards prior to either PET, x-ray CT, or MRI. In general, SPECT requires isotopes that decay by electron capture and/or gamma emission. Examples of viable SPECT isotopes include, but are not limited to, 123-iodine (¹²³I) and 99m-technetium (^{99m}Tc). Subjects are injected with a radioactively labeled agent, typically at tracer doses. The nuclear decay resulting in the emission of a single gamma ray which passes through the tissue and is measured externally with a SPECT camera. The uptake of radioactivity reconstructed by computers as a tomogram shows tissue distribution in cross-sectional images.

Positron emission tomography (PET) is a technique for measuring the concentrations of positron-emitting isotopes within the tissues. Like SPECT, these measurements are, typically, made using PET cameras outside of the living subjects. PET can be broken down into several steps including, but not limited to, synthesizing a compound to include a positron-emitting isotope; administering the isotopically labeled compound to a mammal; and imaging the distribution of the positron activity as a function of time by emission tomography. PET is described, for example, by Alavi et al. in Positron Emission Tomography. published by Alan R. Liss, Inc. in 1985.

Positron-emitting isotopes used in PET include, but are not limited to, Carbon-11, Nitrogen-13, Oxygen-15, and Fluorine-18. In general, positron-emitting isotopes should have short half-lives to help minimize the long-term radiation exposure that a patient receives from high dosages required during PET imaging.

In certain instances, PET imaging can be used to measure the binding kinetics of compounds of this invention with 5-HT₆ serotonin receptors. For example, administering an isotopically labeled compound of the invention that penetrates into the body and binds to a 5-HT₆ serotonin receptor creates a baseline PET signal which can be monitored while administering a second, different, non-isotopically labeled compound. The baseline PET signal will decrease as the non-isotopically labeled compound competes for the binding to the 5-HT₆ serotonin receptor.

In general, compounds of formula I that are useful in performing PET or SPECT are those which penetrate the blood-brain barrier, exhibit high selectivity, high affinity to 5-HT₆ serotonin receptors, and are eventually metabolized. Compounds that are non-selective or those that exhibit excessive or limited affinity for 5-HT₆ serotonin receptors are, generally, not useful in studying brain receptor binding kinetics with respect to 5-HT₆ serotonin receptors. Compounds that are not metabolized may pose safety risks. A mammal is injected with a radioactively labeled agent at tracer doses. Tracer doses are doses sufficient to allow the receptor occupancy to be measured (e.g., to allow detection of the labeled compound) but are not sufficient to have a therapeutic effect on the mammal. Tracer dosage is generally between approximately 1/100 to approximately 1/10 of the therapeutic dose. The radiolabeled compound of formula I is generally administered once daily and is generally administered intravenously.

In other embodiments, nuclear magnetic resonance spectroscopy (NMR) imaging can be used to detect the overall concentration of a compound or fragment thereof containing nuclei with a specific spin. In general, the isotopes useful in NMR imaging include, but are not limited to, hydrogen-1, carbon-13, phosphorus-31, and fluorine-19.

Further, substitution with heavier isotopes such as deuterium, i.e., ²H, can afford certain therapeutic advantages resulting from greater metabolic stability, for example increased in vivo half-life or reduced dosage requirements and, hence, maybe preferred in some circumstances. Isotopically labeled compounds of Formula I of this invention can generally be prepared by carrying out the synthetic procedures described above by substituting an isotopically labeled reagent for a non-isotopically labeled reagent.

Preparation of Tert-butyl 4-{3-(acetylamino)-4-[(3-fluorophenyl)sulfonyl]phenyl}-1,4-diazepane-1-carboxylate (Compound 0):

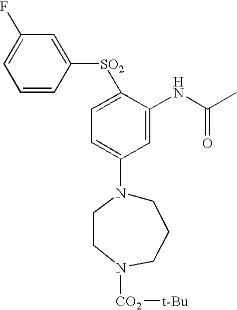

Step 1: To a mixture of 3-fluorothiophenol (8.36 g, 65.2 mmol) and potassium carbonate (9.81 g, 71.0 mmol) in 115 mL of dry acetonitrile is added 2,5-difluoronitrobenzene (7.00 mL, 64.55 mmol). The mixture is allowed to stir at rt 2 hours. Water and CH₂Cl₂ (120 mL each) are added and the layers separated. The aqueous layer is extracted with CH₂Cl₂ (3×50 mL). The combined organics are dried over MgSO₄, filtered, and concentrated to give a bright yellow solid. The solid is triturated with hexane to give 17.1 g of 4-fluoro-2-nitrophenyl 3-fluorophenyl sulfide as a bright yellow solid. ¹H NMR (300 MHz, CDCl₃) δ 7.95, 7.41-7.51, 7.33-7.38, 7.26-7.31, 7.12-7.24, 6.91.

Step 2: A mixture of 4-fluoro-2-nitrophenyl 3-fluorophenyl sulfide (17.1 g, 64.1 mmol) in 430 mL of glacial acetic acid is heated to 100° C. and treated with 7.4 mL of a 30% H₂O₂ solution. After stirring for 20 min at 100° C., another 7.4 mL of the H₂O₂ solution is added and the mixture stirred at 100° C. for an additional 30 min. After cooling to room temperature, the mixture is diluted with 450 mL of water and the resulting white solid is filtered. The solids are rinsed with 1 N NaOH, and water until neutral to litmus, triturated with hexane, and dried under vacuum to give 18.5 g of 4-fluoro-2-nitrophenyl 3-fluorophenyl sulfone as a white solid. ¹H NMR (300 MHz, CDCl₃) δ 8.38-8.43, 7.74-7.77, 7.62-7.66, 7.47-7.60, 7.31-7.48.

Step 3: A mixture of 4-fluoro-2-nitrophenyl 3-fluorophenyl sulfone (5.00 g, 16.7 mmol), homopiperazine (2.09 g, 20.9 mmol), and potassium carbonate (3.46 g, 25.1 mmol) in 140 mL of dry acetonitrile is stirred vigorously for 4 h at 60° C. After cooling to rt, water and CH₂Cl₂ (140 mL each) are added and the layers separated. The aqueous layer is extracted with CH₂Cl₂ (3×50 mL). The combined organics are dried over MgSO₄, filtered, and concentrated to give an orange solid. The crude product is triturated with a hot Et₂O:CH₂Cl₂ mixture (4:1) until solid. Further trituration with hot EtOAc/hexane gives 6.31 g of 4-(1,4-diazepan-1-yl)-2-nitrophenyl 3-fluorophenyl sulfone as bright yellow solid. ¹H NMR (300 MHz, CDCl₃) δ 8.05, 7.71-7.76, 7.59-7.64, 7.46-7.54, 7.22-7.30, 6.90, 6.83, 3.54-3.70, 3.00-3.07, 2.80-2.88, 1.82-1.94.

Step 4: To a mixture of 4-(1,4-diazepan-1-yl)-2-nitrophenyl 3-fluorophenyl sulfone (49.1 g, 129 mmol) and sodium hydroxide (11.4 g, 286 mmol) in 650 ml of a 1:1 THF:H₂O solvent is slowly added a mixture of di-tert-butyl dicarbonate (32.4 g, 148 mmol) in 5 ml of THF. The solution is stirred at rt for 16 h. The mixture is neutralized with 6 N HCl and 200 mL of CH₂Cl₂ is added. The layers are separated and the aqueous layer extracted with CH₂Cl₂ (3×100 mL). The combined organics are dried over MgSO₄, filtered, and concentrated to give an orange solid. The crude product is triturated with EtOAc and hexane to give 60.35 g of tert-butyl 4-[4-[(3-fluorophenyl)sulfonyl]-3-nitrophenyl]-1,4-diazepane-1-carboxylate as a bright yellow solid. ¹H NMR (400 MHz, CDCl₃) δ 8.12, 7.76-7.80, 7.63-7.67, 7.52-7.58, 7.31-7.34, 6.94-6.97, 6.87-6.92, 3.62-3.75, 3.39-3.45, 3.30-3.35, 1.94-2.03, 1.42, 1.34.

Step 5: A mixture of tert-butyl 4-{4-[(3-fluorophenyl)sulfonyl]-3-nitrophenyl}-1,4-diazepane-1-carboxylate (58.0 g, 121 mmol) and Pd/C (8.70 g, 15% by wt.) in 1.5 L of a 2:2:1 THF:MeOH:EtOH solvent is exposed to hydrogen gas (25 psi) in a Parr bottle. The pressure of hydrogen is constantly monitored and kept near 25 psi. After 16 h, the mixture is filtered, solids rinsed with MeOH and CH₂Cl₂, and filtrate concentrated to give a brown solid. The solid is triturated with EtOAc and hexane to give 53.4 g of tert-butyl 4-[3-amino-4-[(3-fluorophenyl)sulfonyl]phenyl-]1,4-diazepane-1-carboxylate as an off white solid. ¹H NMR (400 MHz, CDCl₃) δ 7.72-7.76, 7.64, 7.44-7.51, 7.21-7.27, 6.22-7.26, 5.84-5.87, 5.03-5.09, 3.52-3.63, 3.33-3.38, 3.23-3.29, 1.91-1.99, 1.45, 1.34.

Step 6: Compound 0

A mixture of tert-butyl 4-[3-amino-4-[(3-fluorophenyl)sulfonyl]phenyl]-1,4-diazepane-1-carboxylate (0.29 g, 0.66 mmol) and anhydrous acetic anhydride (1.0 mL) is stirred at 60° C. for 1 h under a nitrogen atmosphere. After cooling to rt, 5 mL of toluene is added and the mixture concentrated. The crude product is triturated with more toluene then with EtOAc and Et₂O to give 0.31 g of Compound 0 as a white solid. ¹H NMR (400 MHz, CDCl₃) δ 9.50-9.66, 7.78-7.85, 7.67, 7.51-7.54, 7.37-7.45, 7.14-7.19, 6.35-6.44, 3.50-3.58, 3.21-3.27, 3.12-3.17, 2.15, 1.86-1.94, 1.33, 1.28.

### EXAMPLE 1

### N-{5-(1,4-diazepan-1-yl)-2-[(3-fluorophenyl)sulfonyl]phenyl}acetamide Methanesulfonate

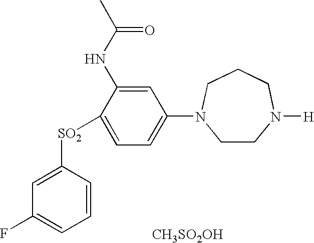

A solution of Compound 0 (2.64 g, 5.36 mmol) in CH₂Cl₂ (50 mL) and Et₂O (50 mL) is treated with methanesulfonic acid (0.37 ml, 5.7 mmol). The mixture is stirred for 2.5 h and concentrated to provide 2.17 g as an off white solid. A portion of the crude solid (0.311 g) is crystallized (4:1-EtOAc:acetone) to afford 0.241 g of Example 1 as an off-white crystalline solid: ¹H NMR (400 MHz, CDCl₃) δ 7.94, 7.83, 7.60-7.70, 7.50-7.60, 7.25-7.40, 6.50, 3.85-3.95, 3.67-3.75, 3.35-3.45, 3.15-3.25, 2.74, 2.30-2.40, 2.27.

### EXAMPLE 2

### N-{5-(1,4-diazepan-1-yl)-2-[(3-fluorophenyl)sulfonyl]phenyl}acetamide Hydrochloride

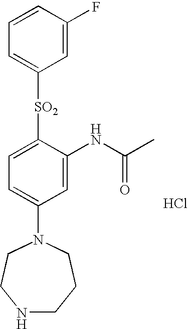

A solution of the freebase of N-{5-(1,4-diazepan-1-yl)-2-[(3-fluorophenyl)sulfonyl]phenyl}acetamide in ethanol 0.19 M (25 mL, 4.8 mmol) is prepared. A solution of acetyl chloride (0.33 mL, 4.6 mmol) in ethanol (˜5 mL) is added to this mixture and the mixture reduced to ˜15 mL. The resulting solid is filtered and dried to give 1.46 g of Example 2 as a white solid (mp 237-238° C.): ¹H NMR (400 MHz, DMSO-d₆) δ 9.45, 9.20, 7.83, 7.60-7.75, 7.45-7.60, 7.19, 6.77, 3.65-3.85, 3.50-3.65, 3.00-3.30, 1.95-2.20.

The above procedure produces a mixture of two crystal forms of N-{5-(1,4-diazepan-1-yl)-2-[(3-fluorophenyl)sulfonyl]phenyl}acetamide hydrochloride. Subsequent heating of the isolated solid containing the two crystal forms to about 70-80° C. under N₂ to reduce the relative humidity of the sample produces a single stable crystal form. The physical characteristics of the single, stable crystal form are listed in Table 2. FIG. 1 shows a plot of a powder X-ray Diffraction spectra of N-{5-(1,4-diazepan-1-yl)-2-[(3-fluorophenyl)sulfonyl]phenyl}acetamide hydrochloride salt as the stable crystalline form.

### EXAMPLE 3

### N-{5-(1,4-diazepan-1-yl)-2-[(3-fluorophenyl)sulfonyl]phenyl}acetamide Trifluoroacetate

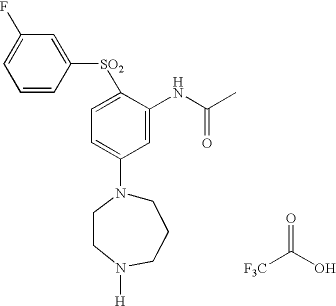

A solution of Compound 0 (1.00 g, 2.00 mmol) in CH₂Cl₂ (20 mL) is cooled in an ice bath and subsequently treated with trifluoroacetic acid (20 mL). After 2 h, the mixture is diluted with toluene (20 mL) and concentrated. The resulting foam is crystallized (EtOAc/hexane) to afford 0.77 g of Example 3 as a white solid: ¹H NMR (400 MHz, CDCl₃) δ 9.85, 7.85-7.95, 7.80-7.85, 7.60-7.70, 7.45-7.60, 7.25-7.35, 3.85-3.95, 3.65-3.75, 3.25-3.40, 3.10-3.25, 2.20-2.45.

### EXAMPLE 4

### N-{5-(1,4-diazepan-1-yl)-2-[(3-fluorophenyl)sulfonyl]phenyl}acetamide Maleate, Crystal Form I

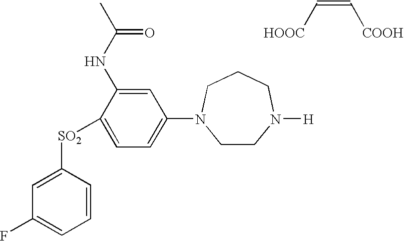

Maleic acid (0.541, 4.65 mmol) is added to a 0.19 M solution of the freebase of N-{5-(1,4-diazepan-1-yl)-2-[(3-fluorophenyl)sulfonyl]phenyl}acetamide in ethanol (25 mL, 4.8 mmol). The mixture is concentrated to an oil and re-dissolved with heating in ethanol (˜15 ml). Upon cooling, the solid is collected to afford 1.92 g of Example 4 as a white solid (mp 176-178° C.): ¹H NMR (400 MHz, DMSO-d₆) δ 9.43, 8.50-8.70, 7.83, 7.60-7.75, 7.50-7.60, 7.19, 6.77, 6.02, 3.65-3.80, 3.45-3.60, 3.20-3.35, 3.05-3.20, 1.95-2.15.

FIG. 1A is a powder X-ray Diffraction spectrum of Example 4.

### EXAMPLE 5

### N-{5-(1,4-diazepan-1-yl)-2-[(3-fluorophenyl)sulfonyl]phenyl}acetamide Maleate, Crystal Form I & Crystal Form II

A solution of maleic acid (4.71, 40.6 mmol) in ethanol (50 mL) is added to a 0.52 M solution of the freebase of N-{5-(1,4-diazepan-1-yl)-2-[(3-fluorophenyl)sulfonyl]phenyl}acetamide in ethanol (80 mL, 41.4 mmol). The resulting solution is concentrated partially at rt and the resulting solid is collected and dried to afford 19.0 g, of Example 5 as a white solid (mp 169-171° C.): ¹H NMR (400 MHz, DMSO-d₆) δ 9.43, 8.50-8.70, 7.83, 7.60-7.75, 7.50-7.60, 7.19, 6.77, 6.02, 3.65-3.80, 3.45-3.60, 3.20-3.35, 3.05-3.20, 1.95-2.15.

FIG. 2B is a powder X-ray Diffraction spectrum of a mixture of Crystal Form I and Crystal Form II of N-{5-(1,4-diazepan-1-yl)-2-[(3-fluorophenyl)sulfonyl]phenyl}acetamide maleate. Recrystallization of this mixture can produce either crystal form I or crystal form II depending upon the recrystallization solvent and conditions. Table I summarizes the recrystallization of mixtures of crystalline forms I and II of N-{5-(1,4-diazepan-1-yl)-2-[(3-fluorophenyl)sulfonyl]phenyl}acetamide maleate for producing crystalline forms I or II alone, or a mixture of crystalline forms I and II. FIG. 2C is a powder X-ray Diffraction spectrum of crystal form II.

Each recrystallization experiment is conducted by dissolving approximately 30-50 mg of N-{5-(1,4-diazepan-1-yl)-2-[(3-fluorophenyl)sulfonyl]phenyl}acetamide maleate crystal forms I and II in several different solvent(s), as specified in Table 1, with stirring. Crystallization occurs under various conditions as specified in Table 1. Analysis of the solid forms of the resulting materials is completed primarily by powder X-ray diffraction and thermal analysis. Additional analytical data (thermal gravimetric analysis, moisture sorption, microscopy) was also collected on selected samples. Non-crystalline materials were not further analyzed. Over twenty recrystallization experiments were conducted to assess potential solid forms of N-{5-(1,4-diazepan-1-yl)-2-[(3-fluorophenyl)sulfonyl]phenyl}acetamide maleate. As described in Table I, most crystallization trials produced crystalline materials. Further examination by powder X-ray Diffraction identified that the recrystallization studies produced two unique crystal forms of the maleate salt. Form I crystalline material is produced from the following solvent systems: ethanol (hot or reflux), benzyl alcohol/methyl t-butyl ether 1:1, isopropanol, and water. Form II crystalline material is produced from the following solvent systems: dioxane, acetone, acetonitrile, and butanol/water 1:1.

TABLE 1
Recrystallization Study

| | | Solid Form | |
|---|---|---|---|
| Solvent* | Appearance** | (XRD)** | Conditions*** |
| EtOH | Xtal | F1 | Warm-cool |
| EtOH | Xtal | F1 + F2 | RT-cool |
| EtOH | Xtal | F1 | Hot-reflux |
| MeOH | Glass | N/R | Hot-evap. |
| MeOH | Sticky solid + | Amp + F1 + F2 | RT-cool |
| | Glass | | |
| THF/H₂O/ | Glass + Sticky | Amp | RT-evap |
| Dioxane | solid | | |
| IPA/H₂O | Glass | N/R | RT-cool |
| Acetone/ | Sticky solid | Amp + F2 + F1 | RT-evap |
| H₂O/EtOAc | | | |
| MeOH/Toluene | Glass | N/R | RT-evap |
| BA | Sticky solid | F2 + Amp | Crush w/MtBE |
| ACN | Xtal | F2 | RT-cool |
| ACN | Xtal + Glass | F2 + Amp | RT-evap |
| ACN/Dioxane | Xtal + Glass | F2 + F1 + Amp | RT-evap |
| Butanol/H₂O | Xtal | F2 | RT-eva |
| Toluene/MeOH | Xtal + Glass | F1 + F2 + Amp | RT-evap |
| ACN | Sticky solid + | F2 + Amp | RT-evap |
| | Glass | | |
| BA/MtBE | Xtal | F1 | Slow addition |
| Acetone | Glass | N/R | RT-cool |
| ACN/H₂O | Glass | N/R | RT-evap |
| MeOH/EtOAc | Glass | N/R | RT-evap |
| IPA | Xtal | F1 | RT-cool |
| Dioxane | Xtal | F2 | RT-cool |
| ACN | Xtal | F2 | Hot-cool |
| MeOH | Sticky solids | Amp | Warm-cool |
| IPA/H₂O | Sticky solids | F2 + Amp | Warm-cool |
| Acetone | Xtal | F2 | Warm-cool |
| Dioxane | Xtal | F2 | Hot-cool |
| EtOH | Xtal | F1 | Hot-cool |
| H₂O | Xtal | F1 | Warm-cool |
| EtOH | Xtal | F1 + Amp | Hot-evap |
| H₂O | Xtal | F1 | Warm-evap |

Notes:
XRD means X-ray Diffraction.
EtOAc means ethyl acetate.
BA means benzyl alcohol.
ACN means acetonitrile.
MtBE means methyl t-butylether.
IPA means isopropyl alcohol.
*Starting Material was a mixture of crystal forms I (F1) and crystal forms II (F2).
**Amp = amorphous, Xtal = crystalline, Glass = glassy material, N/R = not run.
RT means room temperature or ambient temperature (20-25° C.); Warm means 25-35° C.; Hot means >35° C.
***Samples designated from RT-cooling experiments were set in the lab hood at RT overnight, then moved to refrigerator condition at 4° C. for 2-3 days.

Samples designated from hot-cooling experiments were placed on a hot plate inside the lab hood and the solutions were stirred at specified temperature, and then moved to refrigerator condition at 4° C. for 2-3 days.

Samples designated from RT-evaporation were evaporated at RT with gentle nitrogen stream.

Samples designated from hot-evaporation were evaporated at about 40° C. with gentle nitrogen stream.

The crystalline material of forms I and II exhibit similar primary particle morphologies, e.g., rod, blade, and plates, as analyzed via optical microscopic observation. Both crystalline forms I and II also exhibit excellent physical stability upon storage at stressed environment conditions over two weeks at either high temperature (70° C.) or high humidity (88% RH). See results in Table 2 below. The DMSG and DSC data for crystalline form I and form II are shown in FIGS. 2 and 3, respectively. Form I is thermodynamically favored relative to form II as evidenced by conversion of form II to form I upon stirring in aqueous or hot-ethanol.

### EXAMPLE 6

### N-{5-(1,4-diazepan-1-yl)-2-[(3-fluorophenyl)sulfonyl]phenyl}acetamide Hemi-citrate

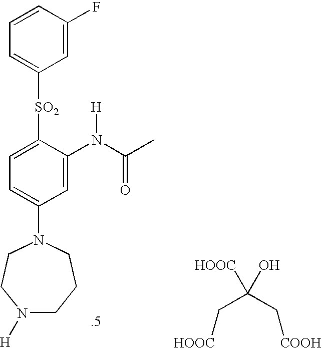

Example 6 is obtained as a white solid using the procedures discussed herein to obtain the salts making non-critical changes using a 0.5:1 stoichiometry (citric acid:amine) in 28% yield (mp 173-176° C.): ¹H NMR (400 MHz, DMSO-d₆) δ 9.40, 7.80, 7.65-7.75, 7.50-7.60, 7.20, 6.72, 3.50-3.75, 3.05-3.15, 2.85-3.00, 2.40-2.60, 2.06, 1.85-1.95.

### EXAMPLE 7

### N-{5-(1,4-diazepan-1-yl)-2-[(3-fluorophenyl)sulfonyl]phenyl}acetamide Hemi-succinate

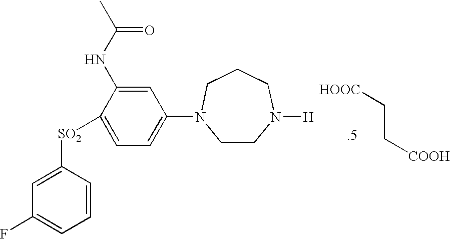

Example 7 is obtained as a white solid using the procedures discussed herein to obtain the salts making non-critical changes using a 0.5:1 stoichiometry (succinic acid:amine) in 12% yield (mp 129-132° C.): ¹H NMR (400 MHz, DMSO-d₆) δ 9.40 (s, 1 H), 7.78, 7.60-7.75, 7.50-7.60, 7.20, 6.69, 3.45-3.60, 2.90-3.00, 2.70-2.80, 2.31, 2.05, 1.70-1.85.

### EXAMPLE 8

### N-{5-(1,4-diazepan-1-yl)-2-[(3-fluorophenyl)sulfonyl]phenyl}acetamide Tosylate

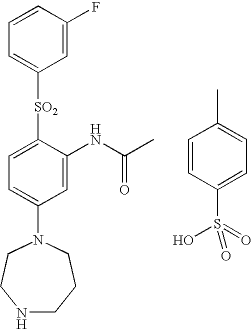

A 0.07 M solution of the freebase of N-{5-(1,4-diazepan-1-yl)-2-[(3-fluorophenyl)sulfonyl]phenyl}acetamide in ethanol (20 mL, 1.41 mmol) is treated with p-toluenesulfonic acid monohydrate (0.269 g, 1.41 mmol). The resulting solution is concentrated and the residue crystallized (IPA/acetone) to give 0.246 g of Example 8 as a white solid; ¹H NMR (400 MHz, DMSO-d₆) δ 8.60, 7.83, 7.60-7.75, 7.50-7.60, 7.46, 7.19, 7.11, 6.75, 3.65-3.75, 3.45-3.55, 3.20-3.30, 3.10-3.20, 2.29, 1.95-2.10.

### EXAMPLE 9

### N-{5-(1,4-diazepan-1-yl)-2-[(3-fluorophenyl)sulfonyl]phenyl}acetamide Hemi-fumarate

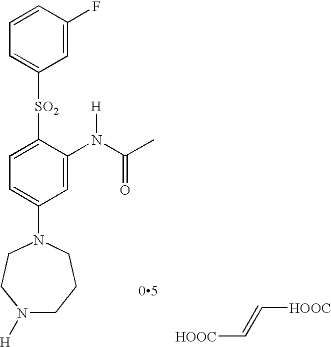

Example 9 is obtained as a white solid using the procedures discussed herein to obtain the salts making non-critical changes using a 0.5:1 stoichiometry (fumaric acid:amine) (mp 151-155° C.): ¹H NMR (400 MHz, DMSO-d₆) δ 9.38, 7.78, 7.60-7.75, 7.45-7.60, 7.20, 6.70, 6.44, 3.45-3.60, 2.85-3.00, 2.70-2.85, 2.05, 1.75-1.90.

### EXAMPLE 10

### N-{5-(1,4-diazepan-1-yl)-2-[(3-fluorophenyl)sulfonyl]phenyl}acetamide Hemi-malate

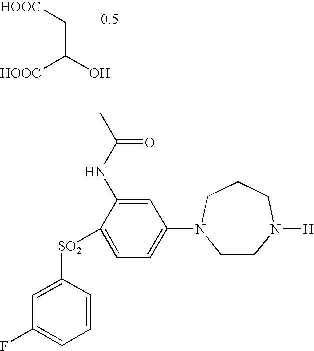

Example 10 is obtained as a white solid using the procedures discussed herein to obtain the salts making non-critical changes using a 1:1 stoichiometry (malic acid:amine) (mp 157-160° C.): ¹H NMR (400 MHz, DMSO-d₆) δ 9.40, 7.79, 7.60-7.75, 7.50-7.60, 7.20, 6.71, 3.82, 3.45-3.65, 3.17, 2.90-3.10, 2.75-2.90, 2.30, 2.25-2.35, 2.06, 1.75-1.90.

### EXAMPLE 11

### N-{5-(1,4-diazepan-1-yl)-2-[(3-fluorophenyl)sulfonyl]phenyl}acetamide Hemi-tartrate

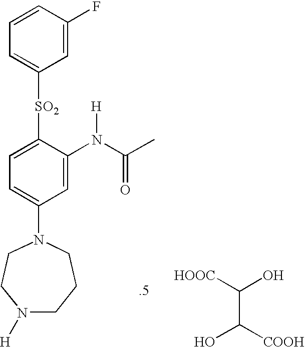

Example 11 is obtained as a white solid using the procedures discussed herein in the salts making non-critical changes using a 0.5:1 stoichiometry (tartaric mine) (mp 196-198° C.): ¹H NMR (400 MHz, DMSO-d₆) δ 9.40, 7.79, 7.55.45-7.55, 7.20, 6.72, 3.82, 3.45-3.65, 2.95-3.10, 2.80-2.95, 2.05, 1.80-1.95.

TABLE 2
Characterization & Stability Summary for Salts

| | | | DSC | | |
|---|---|---|---|---|---|
| | Δ-Wt (%) | | (° C.) | Stability at | Stability at |
| Exam- | DMSG: 0- | | (peak | high RH | high Temp |
| ple | 90% RH | XRD | temp) | (88%)* | (70° C.) |
| 1 | Deliquesced | Crystalline | 137.0 | Deliquesced | Stable |
| | (>70% RH) | | | (>70% RH) | |
| 2 | 0.8 | Crystalline | 243.0 | Stable | Stable |
| 3 | 0.8 | Crystalline | 181.5 | Stable | Stable |
| 4 | 0.7 | Crystalline | 179.0 | Stable | Stable |
| 5 | 0.3 | Crystalline | 161.4 | Stable | Stable |
| (Form | | | | | |
| II) | | | | | |
| 6 | 2.5 | Crystalline | 176.7 | Unstable*** | Stable |
| 7 | 5.0 | Crystalline | 130.4 | Unstable** | Unstable** |
| 8 | 1.9 | Crystalline | 168.3 | Stable | Stable |
| 9 | 3.0 | Crystalline | 143.6, | Unstable** | Stable |
| | | | 158.9 | | |
| 10 | 1.5 | Crystalline | 158.5 | Stable | Unstable** |
| 11 | 7.0 | Crystalline | 199.2 | Unstable** | Stable |

*Preliminary assessment indicated that nine out of the ten salts (except the mesylate salt) remain in solid phase at high RH conditions.
**Two-week stability assessment by Powder-XRD had shown that the original solid sample had converted to a new solid form.
***Two-week stability assessment by Powder-XRD had shown that part of the original solid sample had converted to a new solid form.

Characterization and stability assessments (Table II) indicates that four salts (HCL, maleate, TFA and tosylate) unexpectedly exhibit better physical stability at stressed conditions (high temperature and/or high humidity) and lower moisture absorption (Δ-Wt(%) DMSG) than other salts.

In the preceding examples, ¹H NMR are recorded on either a Bruker 300 MHz or 400 MHz instrument in the indicated solvent. Chemical shifts are expressed in parts per million (δ) using residual solvent protons as an internal standard. Melting points were observed using a Buchi 545 capillary melting point apparatus and are uncorrected. Analytical thin layer chromatography (TLC) analyses were performed on pre-coated EM Science 250 μm silica gel 60 F₂₅₄ plates in the appropriate solvent systems. Developed plates were visualized under UV light. Column chromatography was performed on EM Science silica gel 60.

The physical characteristics of each example were tested by recording X-ray Diffraction (XRD) Analysis, Differential scanning calorimetry (DSC), and Dynamic moisture sorption gravimetry (DMSG).

X-ray Diffraction (XRD) Analysis: A Scintag X-2 theta/theta scanning diffractometer (Scintag Inc., Cupertino Calif.) was employed for the XRD data acquisition for this study. The Sintag X-2 X-ray diffractometer uses a copper tube with K-L₃ radiation at 1.54016 Angstrom (also known as the Cu-Kα₁ emission). Major instrumental parameters are set as follows: 45 KV voltage, 40 mA current, divergent slit width of 2 mm, detector scatter slit width of 0.5 mm and receiving slit width of 0.3 mm. Each XRD pattern was scanned over the spectral range of 2-40° two-theta angle with a step scan algorithm with a step size of 0.03° two-theta angle/step. The raw XRD data, exported in ASCII format, is then converted to the binary format with Galactic's Grams software (Galactic Industries Corp., Salem N.H.) for data analysis, storage and output.

Differential scanning calorimetry (DSC): DSC was performed using a TA Instruments model 2920 module with a Thermal Analyst 5000 controller (TA Instruments, Wilmington Del.). Data were collected and analyzed using TA Instruments Thermal Solutions for NT and Universal Analysis for NT software. A sample of about 1-mg was accurately weighed into an aluminum pan with lid, which was crimped to ensure good thermal contact. The sample was heated using a linear ramp rate of 10° C./min from ambient to approximately 300° C.

Dynamic moisture sorption gravimetry (DMSG): DMSG isotherms were collected on the variable temperature controlled atmospheric microbalance, CAM. Approximately 10 mg samples were used in the balance, samples were run as received. The humidity was set at ambient conditions on the day analysis began. After the first scan with fast scan rate from ambient RH to 0% RH; the DMSG analysis followed with three more scans: 0% RH to 90% RH, 90% RH to 0% RH, 0% RH to 90% RH. The scan rate was 3% RH/step. The mass was measured every two minutes and an equilibrium window of 5 points within 0.001 mg. The RH was changed to the next value (+/−3% RH) when the mass of the sample was stable to within 1.0 μg. The Visual Basic program dmsgscn2.exe was used to control the data collection and export the information to an Excel spreadsheet.

What is claimed:

1. A compound of formula I 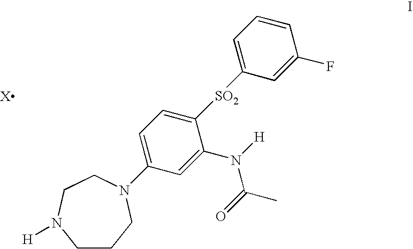
wherein X is selected from HCl, HOC(O)CF₃, cis-HO(CO)—CH═CH—(CO)OH, and

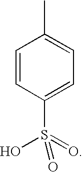

2. The compound of claim 1, wherein X is HOC(O)CF₃.

3. The compound of claim 1, wherein X is cis-HO(CO)—CH═CH—(CO)OH.

4. The compound of claim 3, wherein the compound is crystalline.

5. The compound of claim 4, wherein the compound is crystalline form I.

6. The compound of claim 4, wherein the compound is crystalline form II.

7. The compound of claim 1, wherein X is

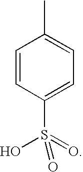

8. The compound of claim 1, wherein X is HCl.

9. The compound of claim 1, wherein the compound of formula I is detectably labeled with a radioisotope.

10. The compound of claim 9, wherein the radiolabel is Carbon-11, Nitrogen-13, Oxygen-15, and Fluorine-18.

11. A method for treating a disease or disorder of the central nervous system in a mammal comprising administering a therapeutically effective amount of a compound of claim 1.

12. The method of claim 11, wherein X is HOC(O)CF₃.

13. The method of claim 11, wherein X is cis-HO(CO)—CH═CH—(CO)OH.

14. The method of claim 11, wherein the compound is crystalline.

15. The method of claim 14, wherein the compound is crystalline form I.

16. The method of claim 14, wherein the compound is crystalline form II.

17. The method of claim 11, wherein X is

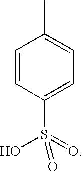

18. The method of claim 11, wherein X is HCl.

19. The method of claim 11, wherein the disease or disorder is selected from psychosis; paraphrenia; depression; psychotic depression; immune system depression; mania; schizophrenia; schizophreniform disorders; schizoaffective disorder; anxiety; migraine headache; cluster headache; drug addiction; convulsive disorders; personality disorders; post-traumatic stress syndrome; alcoholism; panic attacks; panic disorder; obsessive-compulsive disorders; sleep disorders; psychotic, affective, vegetative, and psychomotor symptoms of schizophrenia; extrapyramidal motor side effects of other antipsychotic drugs; eating behavior; obesity; delusional disorder; stress related disease; phobia; a stress induced problem with the urinary, gastrointestinal or cardiovascular system; neurodegenerative disorders; autism; chemotherapy-induced vomiting; hypertension; sexual dysfunction in a mammal; addictive disorder and withdrawal syndrome; adjustment disorder; age-associated learning and mental disorder; apathy; attention-deficit disorder due to general medical conditions; attention-deficit hyperactivity disorder; behavioral disturbance; bipolar disorder; chronic fatigue syndrome; conduct disorder; cyclothymic disorder; dysthymic disorder; somatoform disorders; inhalation disorder; intoxication disorder; movement disorder; oppositional defiant disorder; peripheral neuropathy; premenstrual dysphoric disorder; psychotic disorder; mood disorder; seasonal affective disorder; sleep disorder; cognitive disorders; irritable bowel syndrome; developmental disorder; agitation disorder; SSRI “poop out” syndrome; or Tic disorder.

20. The method of claim 19, wherein the disease is depression.

21. The method of claim 11, wherein said compound is administered in an amount from about 0.01 to about 300 mg/kg of body weight of said mammal per day.

22. A method for treating a disease or disorder in a mammal wherein the 5-HT receptor is implicated and modulation of 5-HT function is desired comprising administering a therapeutically effective amount of a compound of claim 1 to said mammal.

23. The method of claim 22, wherein the receptor is a 5-HT₆ receptor.

24. The method of claim 22, wherein the mammal is a human.

25. The method of claim 22, wherein X is cis-HO(CO)—CH═CH—(CO)OH.

26. The method of claim 25, wherein the compound is crystalline.

27. The method of claim 26, wherein the compound is crystalline form I.

28. The method of claim 26, wherein the compound is crystalline form II.

29. The method of claim 22, wherein said compound is administered in an amount from about 0.01 to about 300 mg/kg of body weight of said mammal per day.

30. The method of claim 22, wherein said compound is administered in an amount from about 1 to about 30 mg/kg of body weight of said mammal per day.

31. The method of claim 22, wherein the disease or disorder is selected from psychosis; paraphrenia; depression; psychotic depression; immune system depression; mania; schizophrenia; schizophreniform disorders; schizoaffective disorder; anxiety; migraine headache; cluster headache; drug addiction; convulsive disorders; personality disorders; post-traumatic stress syndrome; alcoholism; panic attacks; panic disorder; obsessive-compulsive disorders; sleep disorders; psychotic, affective, vegetative, and psychomotor symptoms of schizophrenia; extrapyramidal motor side effects of other antipsychotic drugs; eating behavior; obesity; delusional disorder; stress related disease; phobia; a stress induced problem with the urinary, gastrointestinal or cardiovascular system; neurodegenerative disorders; autism; chemotherapy-induced vomiting; hypertension; sexual dysfunction in a mammal; addictive disorder and withdrawal syndrome; adjustment disorder; age-associated learning and mental disorder; apathy; attention-deficit disorder due to general medical conditions; attention-deficit hyperactivity disorder; behavioral disturbance; bipolar disorder; chronic fatigue syndrome; conduct disorder; cyclothymic disorder; dysthymic disorder; somatoform disorders; inhalation disorder; intoxication disorder; movement disorder; oppositional defiant disorder; peripheral neuropathy; premenstrual dysphoric disorder; psychotic disorder; mood disorder; seasonal affective disorder; sleep disorder; cognitive disorders; irritable bowel syndrome; developmental disorder; agitation disorder; SSRI “poop out” syndrome; or Tic disorder.

32. The method of claim 31, wherein the disease is depression.

33. A pharmaceutically acceptable composition comprising a compound of claim 1 and a pharmaceutically acceptable carrier.

34. The composition of claim 33, wherein the compound is compound of claim 2.

35. The composition of claim 33, wherein the compound is compound of claim 3.

36. The composition of claim 33, wherein the compound is compound of claim 5.

37. The composition of claim 33, wherein the compound is compound of claim 7.

38. The composition of claim 33, wherein the compound is compound of claim 8.

39. A method for diagnosing disease in a mammal, comprising administering to the mammal a detectably labeled compound of claim 9 and detecting binding of said compound to a 5-HT₆ serotonin receptor.

40. The method of claim 39, wherein the compound is detected using position emission topography.

41. The method of claim 39, wherein the compound is detected using single-photon emission computed tomography.
